(19) United States
(12) Patent Application Publication
(10) Pub. No.: US 2014/0012086 A1
(43) Pub. Date: Jan. 9, 2014

(54) SELF-PROPELLING DEVICE

(71) Applicant: FUJIFILM Corporation, Tokyo (JP)

(72) Inventors: Shinichi YAMAKAWA, Ashigarakami-gun (JP); Tsuyoshi ASHIDA, Ashigarakami-gun (JP); Takayuki NAKAMURA, Ashigarakami-gun (JP); Yasunori OHTA, Ashigarakami-gun (JP); Timothy P. SHERIDAN, Eagan, MN (US)

(73) Assignee: FUJIFILM Corporation, Tokyo (JP)

(21) Appl. No.: 14/028,047

(22) Filed: Sep. 16, 2013

Related U.S. Application Data
(63) Continuation of application No. PCT/JP2012/051224, filed on Jan. 20, 2012.
(60) Provisional application No. 61/453,410, filed on Mar. 16, 2011.

(51) Int. Cl. A61B 1/00

(52) U.S. Cl. CPC A61B 1/00156
USPC 600/114

(57) ABSTRACT

A self-propelling device includes a turning unit and a supporter. The supporter includes a first support sleeve, a second support sleeve and a cover formed respectively cylindrically. A drive wheel is secured to the first support sleeve in a rotatable manner. A pair of follower rollers are secured to the second support sleeve together. The cover is fitted on an outer surface of the second support sleeve externally, and disposed in the turning unit together with the second support sleeve. The turning unit is pressed by the follower rollers to the drive wheel. When the drive wheel rotates, the turning unit is circulated around the second support sleeve in a longitudinal direction of the first support sleeve. An inner wall of a body cavity will not be damaged, as the follower rollers are covered by the cover and do not appear externally even if the turning unit breaks.

### BACKGROUND OF THE INVENTION

1. Field of the Invention

The present invention relates to a self-propelling device of a self-running type for guiding entry of an endoscope into a body cavity.

2. Description Related to the Prior Art

An introducer of an endoscope for entry in a body cavity of a patient includes a short rigid tip portion, a bendable portion and a flexible portion with a control handle. The rigid tip portion is disposed at a distal end of the introducer. The bendable portion is formed to extend at a proximal end of the rigid tip portion in a bendable manner for directing the rigid tip portion in a desired direction. The flexible portion is formed at a proximal end thereof in a long form (as long as 1-2 m which differ for the use). A front surface of the rigid tip portion has a viewing window for receiving an image of an object of interest, and the like.

Manipulation for entry of the introducer of the endoscope in a body cavity, for example large intestine, is very difficult, for the reason of the tortuous shape of the large intestine in the body cavity. Considerable experiences are required for learning the manipulation of entry of the endoscope into the large intestine. If the skill for the entry is poor, acute pain will be felt by patients.

A self-propelling device of a self-running type is known, which is mounted on the rigid tip portion of the introducer of the endoscope, and in which a turning unit (called “toroid”) is formed in a doughnut form from soft material of a sheet form and disposed externally (see U.S. Pat. Nos. 6,971,990 and 7,736,300 (corresponding to JP-A 2009-513250)).

The turning unit is nipped between a drive wheel and a follower roller, the drive wheel being disposed on a first supporter disposed outside a coupling member for mounting on the rigid tip portion of the introducer, the follower roller being disposed on a second supporter positioned in an inner space of the turning unit. When the drive wheel is rotated and driven, the turning unit is turned in circulation around the second supporter by continuous movement from an inner side to an outer side of a central cavity (central hole) defined by the turning unit. The self-propelling device guides the introducer of the endoscope into a deep portion of the gastrointestinal tract by propulsion force created by contact of an outer surface of the turning unit on a wall of the gastrointestinal tract and by turning of the turning unit in the circulation.

As the turning unit is formed from soft material of the sheet form, the turning unit may break should high load be applied thereto while driven in the body cavity. Upon the breakage of the turning unit, the follower roller of the second supporter may be exposed in the turning unit, and supposedly will contact and damage an inner wall of the body cavity.

### SUMMARY OF THE INVENTION

An object of the present invention is to provide a self-propelling device in which an inner wall of a body cavity can be prevented from being damaged with a follower roller even when a turning unit is broken.

In order to achieve the above and other objects and advantages of this invention, a self-propelling device of the present invention includes a cylindrical coupling member, a first supporter, a turning unit, a second supporter, a follower roller, a cover and a drive wheel. The cylindrical coupling member is for mounting on a tip portion of an endoscope . The first supporter is formed cylindrically, and fitted on an outer side of the coupling member. The turning unit is formed from flexible material of a sheet form, for covering an outer surface of the first supporter at least partially. The second supporter is disposed in an inner space defined by an inner surface of the turning unit, for supporting the turning unit from inside. The follower roller is disposed on the second supporter in a rotatable manner, for contacting an inner surface of the turning unit. The cover is fitted on an outer surface of the second supporter, for preventing the follower roller from being exposed outside the second supporter. The drive wheel is disposed on the first supporter, for rotating by nipping the turning unit together with the follower roller, for turning the turning unit to circulate around the second supporter and the cover in a longitudinal direction of the first supporter.

Preferably, the follower roller is secured so as not to project from the outer surface of the second supporter externally. Preferably, the cover is cylindrical to cover the outer surface of the second supporter. Also, preferably, the cover is formed from soft resin.

A plurality of the follower roller are combined in a unit and secured to the second supporter in a removable manner. Also, the turning unit is in a doughnut form or is an endless belt.

In the self-propelling device of the present invention, the cover is fitted on the outer surface of the second supporter, and covers the follower roller. Thus, an inner wall of a body cavity can be prevented from being damaged with the follower roller, as the follower roller will not contact the inner wall of the body cavity even upon breakage of the turning unit during use of the device.

The follower rollers are secured not to project from the outer surface of the second supporter externally. Thus, the cover can be fitted on the outer surface of the second supporter in tight contact. For example, the cover is formed from rather soft resin, and an inner diameter of the cover is set slightly smaller than an outer diameter of the second supporter, so that the cover can be fitted externally on the outer surface of the second supporter even without using a fixing means, such as a screw. As a result, the number of the parts and the number of the steps of assembling can be reduced, for contribution in lowering the cost of the self-propelling device.

A plurality of the follower rollers are unified and can be secured to the second supporter in a removable manner. It is possible easily to secure the follower rollers at the time of assembling and to remove the follower rollers at the time of washing. As a result, the number of the steps of assembling can be reduced, for contribution in lowering the cost of the self-propelling device. Also, labor of the maintenance can be reduced.

### BRIEF DESCRIPTION OF THE DRAWINGS

The above objects and advantages of the present invention will become more apparent from the following detailed description when read in connection with the accompanying drawings, in which:

FIG. 1 is a schematic view illustrating an electronic endoscope;

FIG. 2 is a perspective illustrating a state in which a self-propelling device of an embodiment of the present invention is mounted on a tip portion of an introducer of the electronic endoscope;

FIG. 3 is an exploded perspective illustrating a supporter;

FIG. 4 is a section illustrating the self-propelling device.

### DETAILED DESCRIPTION OF THE PREFERRED EMBODIMENT OF THE PRESENT INVENTION

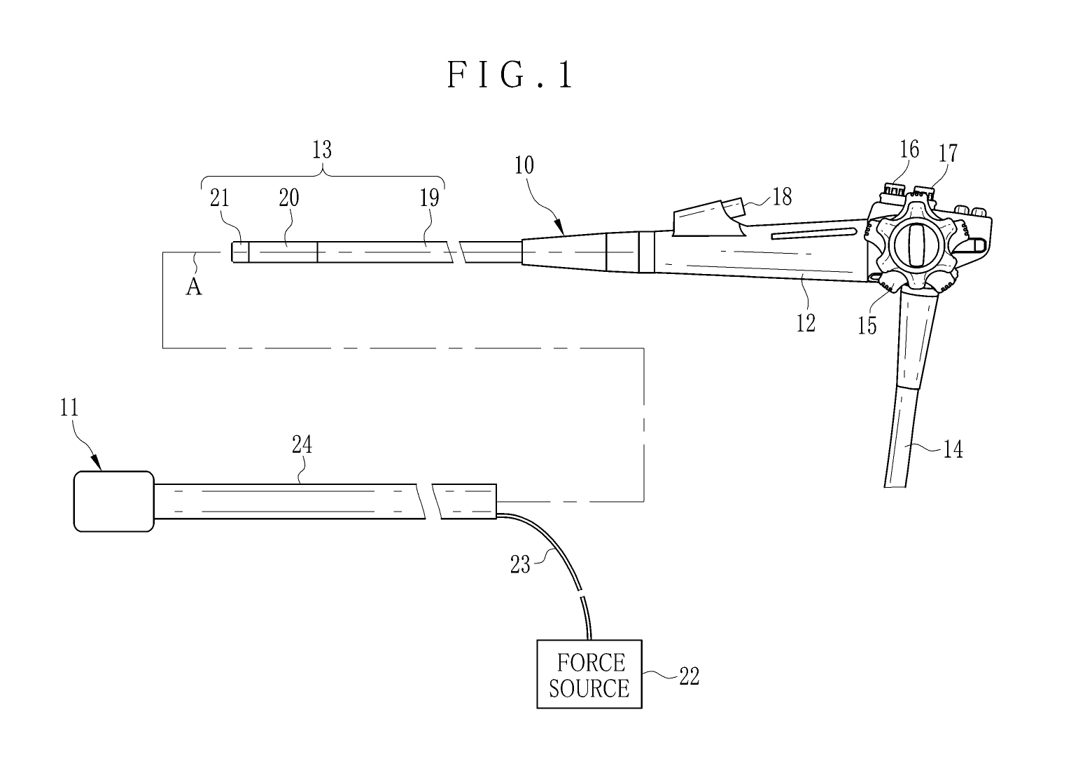

In FIG. 1, an electronic endoscope 10 includes a control handle 12 and an introducer 13 (elongated tube) formed serially with the control handle 12 for entry in a body cavity (for example, large intestine). A universal cord 14 is connected with the control handle 12. Connectors (not shown) are disposed at a tip of the universal cord 14 for connection to respectively a lighting apparatus and a processing apparatus (both not shown) in a removable manner.

The control handle 12 includes angle adjusting knobs 15 (steering wheels), an air/water supply button 16, a suction button 17 and the like. The air/water supply button 16 operates for ejecting air and water through a distal end of the introducer 13. A forceps opening 18 is formed in the control handle 12 on a side of the introducer 13 for entry of a treatment device, such as an electrocautery device.

The introducer 13 includes a flexible portion 19, a bendable portion 20 (steering device) and a rigid tip portion 21 in an order from the control handle 12. The flexible portion 19 has flexibility. The bendable portion 20 has bendability. The flexible portion 19 has a length of for example 1-2 m for the purpose of reach of the rigid tip portion 21 to a target position in the body cavity. The bendable portion 20 is bent up and down and to the right and left by manipulation of the angle adjusting knobs 15 of the control handle 12. Thus, the rigid tip portion 21 can be directed in a desired direction in the body.

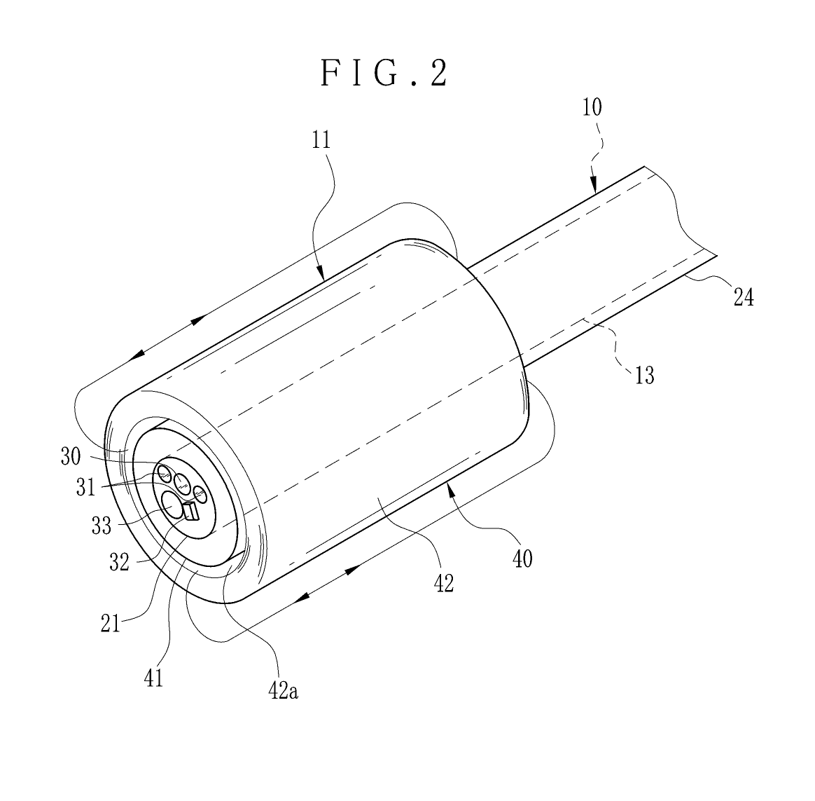

The rigid tip portion 21 includes a viewing window 30 (see FIG. 2), an objective optical system, and a solid state imaging element. The viewing window 30 acquires an image of an object of interest in a body. The solid state imaging element creates an image of the object of interest, such as a CCD or CMOS image sensor. The solid state imaging element is connected to the processing apparatus by a signal cable extended through the introducer 13, the control handle 12 and the universal cord 14. An image of the object of interest is focused on a receiving surface of the solid state imaging element and converted into an image signal. The processing apparatus processes the image signal received through the signal cable from the solid state imaging element in image processing of various functions, to convert the image signal into a video signal. The processing apparatus causes a monitor (not shown) connected by a cable to display an object image of the video signal.

Note that lighting windows 31, an air/water supply nozzle 32 and a forceps outlet 33 are formed in the rigid tip portion (see FIG. 2). The lighting windows 31 apply light for illumination to an object of interest from a light source for illumination in the lighting apparatus. The air/water supply nozzle 32 ejects air or water toward the viewing window after supply from an air/water supply device incorporated in the lighting apparatus. The forceps outlet 33 causes a tip of a treatment device to appear after entry in the forceps opening 18.

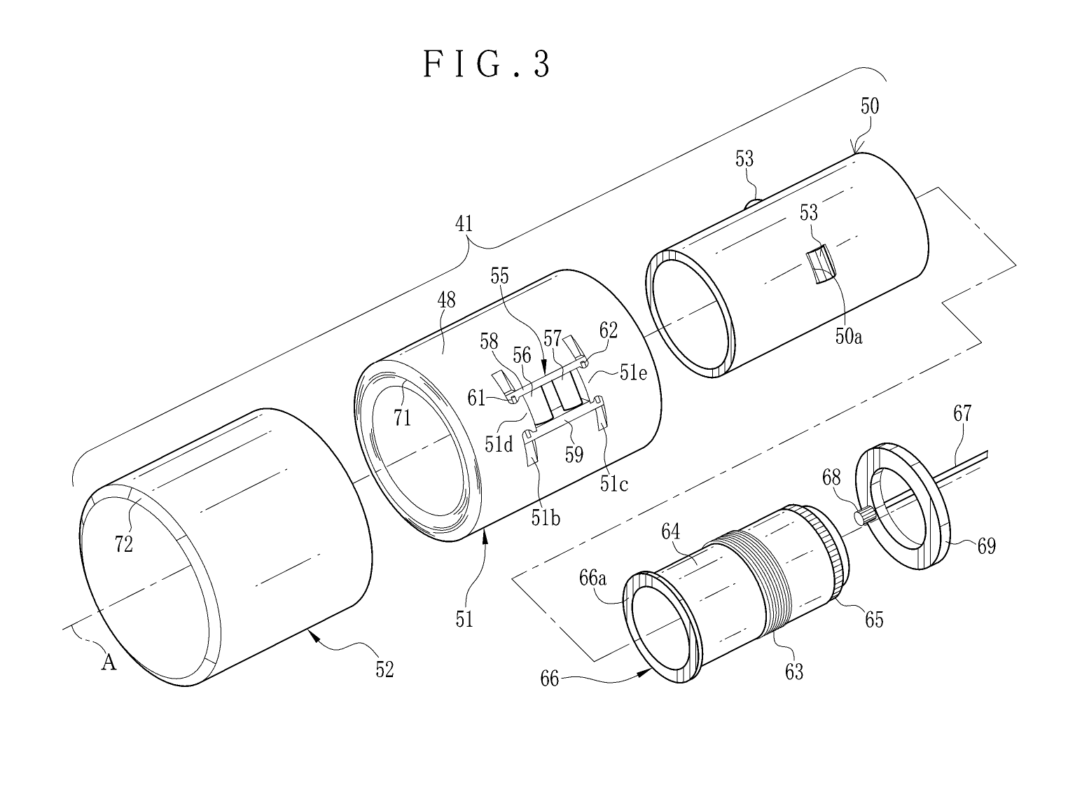

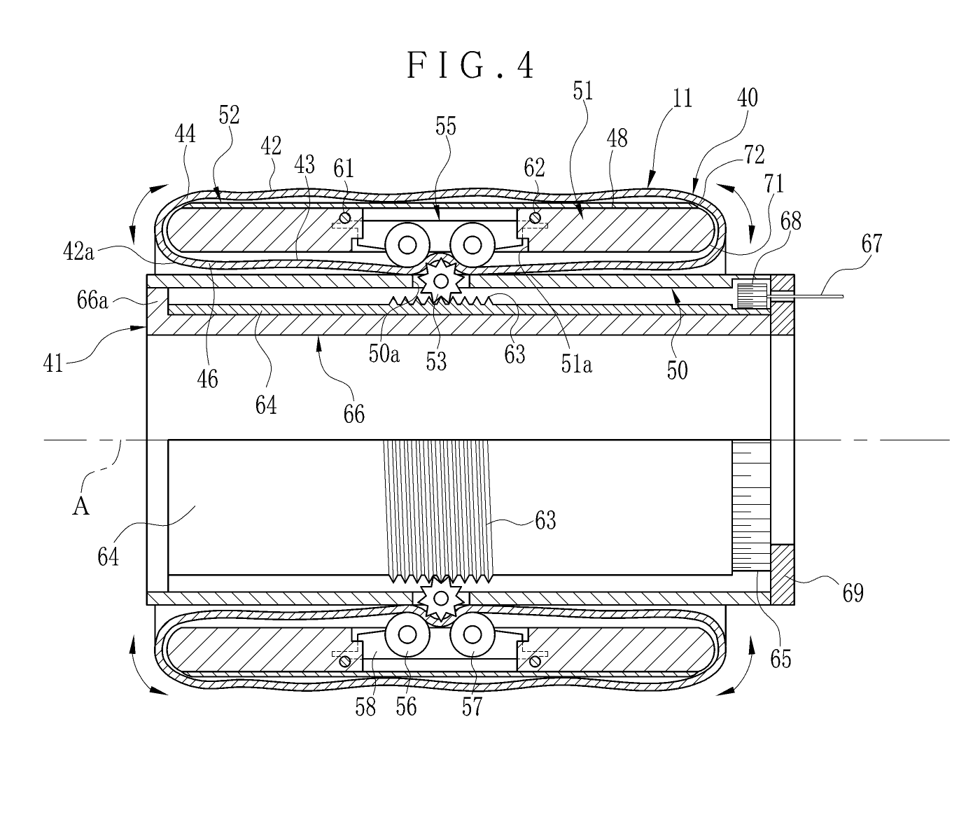

A self-propelling device 11 (propulsion assembly) is a device mounted on the introducer 13 of the electronic endoscope 10 and assists propulsion and return of the introducer 13 within a body cavity. A force source 22 (motor unit) drives the self-propelling device 11. The force source 22 is connected with a torque wire 67 for transmitting rotational torque to drive the self-propelling device 11 (See FIGS. 3 and 4). The torque wire 67 is penetrated through a protection sheath 23 entirely. The torque wire 67 is rotated within the protection sheath 23 by driving of the force source 22. Note that a torque coil can be used in place of the torque wire.

An overtube 24 is fitted externally on the introducer 13, and is extensible in a direction along a longitudinal axis A of the introducer 13. The protection sheath 23 of the torque wire 67 is entered between the overtube 24 and the introducer 13.

The force source 22 includes a motor and a controller (not shown), with which an input unit (not shown) is associated. The input unit includes buttons for inputting instructions of advance, return and stop of the self-propelling device 11, and a speed changing button for changing a moving speed of the self-propelling device 11. Note that a program according to an object of interest may be previously installed, so that the force source 22 can be driven according to the program without manual operation of the input unit, to operate the self-propelling device 11 automatically.

In FIGS. 2-4, the self-propelling device 11 is constituted by a turning unit 40 (endless track device) and a supporter 41 (moving mechanism). The turning unit 40 is similar to, for example, what is called “toroid” in U.S. Pat. Nos. 6,971,990 and 7,736,300 (corresponding to JP-A 2009-513250), and an object in a sheet form made from flexible material, specifically biocompatible plastics such as polyvinyl chloride, polyamide resin, fluorocarbon resin, polyurethane and the like. The turning unit 40 is shaped in a doughnut form extending in the axial direction, and includes an outer surface 42 and an inner surface (internal surface). The turning unit can be called a circulation unit or endless track device.

A central cavity 42a is defined through the turning unit 40. A first support sleeve 50 (first supporter) is disposed inside the central cavity 42a as a constituent of the supporter 41. A return run 46 or lower run (inner side) constituting the central cavity 42a of the turning unit 40 is moved in one direction. A working run 44 or upper run (outer side) not constituting the central cavity 42a of the turning unit 40 is moved in an opposite direction. As a result, the turning unit 40 endlessly moves as indicated by the arrows around the first support sleeve 50 in continuous movement from the inside to the outside or from the outside to the inside. Thus, the working run 44 of the turning unit 40 contacts an inner wall of the body cavity, to create force of propulsion to move the introducer 13 back and forth along the tube axis A.

In FIG. 3, the supporter 41 includes the first support sleeve 50, a second support sleeve 51 (second supporter or barrel sleeve) and a cover 52 in a cylindrical form. The cover 52 according to the present invention is fitted externally on an outer surface 48 of the second support sleeve 51, and disposed in the inner space of the turning unit 40 together with the second support sleeve 51 (See FIG. 4). In FIG. 3, the turning unit 40 is not shown.

The second support sleeve 51 has curved surfaces 71 or first end surfaces, both of which are arcuate and convex as viewed in a cross section. The cover 52 has inclined surfaces 72 or second end surfaces, both of which are formed continuous with the form of the curved surfaces 71 of the second support sleeve 51 without angularly projecting from the second support sleeve 51. Thus, the movement of the turning unit 40 can be smoothed by the extending surfaces from the second support sleeve 51 to the cover 52 with smoothness.

Openings 50a are formed in the first support sleeve 50. Drive wheels 53 are secured in the openings 50a in a rotatable manner in the central portion of the first support sleeve 50 in the direction of the longitudinal axis A. The openings 50a and the drive wheels 53 are three arranged in the circumferential direction of the first support sleeve 50 at a pitch angle of 120 degrees.

Openings 51a are formed in the second support sleeve 51 (see FIG. 4). Follower roller units 55 are fitted in the openings 51ain a removable manner. A pair of follower rollers 56 and 57 are unified to be the follower roller units 55, which include the follower rollers 56 and 57 and a pair of plate portions 58 and 59 (holder portions) formed in a shape of a quadrilateral prism to keep those rotatable in a laterally spaced state. The openings 51a and the follower roller units 55 are three arranged in the circumferential direction of the second support sleeve 51 at a pitch angle of 120 degrees in correspondence with the mount position of the drive wheels 53 in the first support sleeve 50.

To fit the follower roller units 55 in the openings 51a, at first ends of the plate portions 58 and 59 are set down in elongated grooves 51b and 51c which are formed in the second support sleeve 51 in the circumferential direction. Projections 51d and 51e are formed to project from edges of the openings 51a to the inside. Holes of a small diameter are formed in the projections 51d and 51e to penetrate in the circumferential direction. Pins 61 and 62 are entered in respectively the holes. A length of the pins 61 and 62 is greater than a width of the projections 51d and 51e. Ends of the pins 61 and 62 extend beyond the ends of the projections 51d and 51e, to keep ends of the plate portions 58 and 59 inside without rising to the outside of the second support sleeve 51.

In this state, the pins 61 and 62 may drop out of the holes in the projections 51d and 51e by movement. However, the cover 52 is fitted externally on the outer surface of the second support sleeve 51 after fitting three of the follower roller units 55 on the second support sleeve 51. Thus, the pins 61 and 62 are prevented from further movement because their end contacts an inner surface of the cover 52 even when they move slightly. The pins 61 and 62 will not drop. In contrast with this, the pins 61 and 62 are pulled out of the holes of the projections 51d and 51e after removing the cover 52 from the outer surface of the second support sleeve 51, so that the follower roller units 55 can be removed from the openings 51a easily.

When the follower roller units 55 are fitted in the openings 51a, the follower rollers 56 and 57 project slightly from the inner surface of the second support sleeve 51 internally without externally projecting from the outer surface of the second support sleeve 51. The follower rollers 56 and 57 are disposed so that the turning unit 40 is disposed between the drive wheels 53 and those from the sides of the drive wheels 53 in a direction of the longitudinal axis A (See FIG. 4). Thus, the follower rollers 56 and 57 of the pair press the turning unit 40 toward the drive wheels 53.

As the follower rollers 56 and 57 do not project externally from the outer surface of the second support sleeve 51, the cover 52 is formed from resin of rather soft property and has an inner diameter slightly smaller than an outer diameter of the second support sleeve 51. The cover 52 is fitted on the outside of the second support sleeve 51, so that the inner surface of the cover 52 is tightly fitted on the outer surface of the second support sleeve 51. Thus, the cover 52 can be fitted easily, and can be kept without dropping from the second support sleeve 51 even upon incidental offset from the predetermined position in tight contact with the outer surface of the second support sleeve 51.

A cylindrical drive sleeve 64 is disposed inside the first support sleeve 50. A worm gear 63 is formed substantially in the center of the drive sleeve 64. A gear 65 is formed on the drive sleeve 64 from its one end at a predetermined width. A cylindrical coupling member 66 (shaft sleeve) is disposed inside the drive sleeve 64, which is supported on its outer surface in a slidable manner. A pinion 68 (small gear) in connection with the torque wire 67 is meshed with the gear 65. The pinion 68 is rotated by the torque wire 67. Its rotations are transmitted by the gear 65 to the drive sleeve 64, to rotate the worm gear 63 in the circumferential direction.

An annular portion 66a (end ring) is formed at a distal end of the coupling member 66 and has a slightly larger diameter than a main portion of the coupling member 66. A peripheral surface of the annular portion 66a is fitted on an inner surface of the first support sleeve 50 on a distal side. The rigid tip portion 21 of the introducer 13 is entered in and fixed to the inside of the coupling member 66. An example of means for this fixation, which is not shown, is a well-known collet chuck, which includes a sleeve (collet) and a nut, the sleeve having a radial cut extending from its hole center, the sleeve extending on the longitudinal axis A from the edge of the coupling member 66 in an advancing direction (to a distal side of the rigid tip portion 21), the nut having a tapered inner surface and squeezing the outside of the collet for fixation on the rigid tip portion 21. Note that other fixing means can be used.

A rear lid portion 69 (end ring) is fitted on a proximal end of the coupling member 66, keeps the drive sleeve 64 rotatable around the coupling member 66, and prevents the drive sleeve 64 and the first support sleeve 50 from dropping away from the coupling member 66. The rear lid portion 69 is formed at an outer diameter equal to an outer diameter of the first support sleeve 50. A hole is formed in the rear lid portion 69 for penetration of the torque wire 67. The drive sleeve 64, when driven by the pinion 68, rotates about the longitudinal axis A, so that the worm gear 63 formed together with the drive sleeve 64 rotates and drives the drive wheels 53.

Finally, the turning unit 40 is secured to the supporter 41. For the turning unit 40, a material of a sheet form is rolled by attachment of edges. After this, the material is entered between the first support sleeve 50 and the second support sleeve 51 by covering the outside of the first support sleeve 50. At this time, the turning unit 40 of the tubular shape is entered between the first support sleeve 50 and the second support sleeve 51 by rotating the drive wheels 53. A substantially central portion of the turning unit 40 reaches the position of the drive wheels 53. The turning unit 40 is fitted to cover the outside of the cover 52 by bending the edges of the turning unit 40 to the outside. The edges of the turning unit 40 are overlapped on one another at a predetermined width, and attached with adhesive agent or the like. Thus, the turning unit 40 becomes shaped in the doughnut form extending in the axial direction of the central cavity 42a.

The turning unit 40 secured to the supporter 41 is pressed on the drive wheels 53 by the follower rollers 56 and 57. When the drive wheels 53 are rotated and driven, the turning unit 40 turns in circulation in a direction of the longitudinal axis A back or forth (longitudinally with the first support sleeve 50) according to a rotational direction of the worm gear 63 or thus the pinion 68.

The operation of the self-propelling device 11 constructed above is described. At first, the overtube 24 is mounted on the introducer 13 of the electronic endoscope 10. The self-propelling device 11 is mounted on the rigid tip portion 21. After the mount of the overtube 24 and the self-propelling device 11, a power source of the processing apparatus, lighting apparatus, controller and the like is turned on. Then patient information and the like is input. After this, the introducer 13 of the electronic endoscope 10 is entered in a large intestine as a body cavity of a patient.

After the rigid tip portion 21 advances to a predetermined position in the body cavity, for example, short of a sigmoid colon, the input unit is operated to turn on the power source of the force source 22 for the self-propelling device 11. An instruction of the advance is input by button operation of the input unit. The force source 22 (motor unit) rotates the torque wire 67 in a predetermined direction, so the worm gear 63 is rotated by rotations of the pinion 68 together with rotations of the torque wire 67.

Rotations of the worm gear 63 are transmitted to the drive wheels 53, which circulate the turning unit 40 by turning. The outer surface 42 of the turning unit 40 contacts an inner wall of the body cavity, and exerts force for propulsion in the advancing direction. The self-propelling device 11 moves the rigid tip portion 21 forwards along the inner wall of the body cavity as the force of the forward movement of the turning unit 40 pushes the inner wall of the body cavity from a front side to a rear side.

Also, an instruction for changing the speed is input by button operation of the input unit. The force source 22 changes a rotational speed of the torque wire 67. As a result, the moving speed of the self-propelling device 11 is changed. Also, an instruction for backward movement is input by button operation of the input unit. The motor of the force source 22 causes the torque wire 67 to rotate backwards so as to move the turning unit 40 backwards. The self-propelling device 11 is moved backwards to move the rigid tip portion 21 mounted thereon backwards. When an instruction of stop is input by button operation of the input unit, driving of the force source 22 is stopped to stop the self-propelling device 11. Those steps are carried out suitably, so the rigid tip portion 21 can be propelled to a desired position in the body cavity.

While the self-propelling device 11 is used, the turning unit 40 may be broken for reasons of overload and the like. However, the follower rollers 56 and 57 are covered by the cover 52. The follower rollers 56 and 57 will not contact an inner wall of the body cavity and will not damage the soft inner wall. As illustrated in FIG. 4, the curved surfaces 71 and the inclined surfaces 72 of the second support sleeve 51 and the cover 52 are convexly arcuate in a smooth manner as viewed in a cross section, and will not damage an inner wall of the body cavity because they do not have a tapered portion.

If the turning unit 40 is broken, a grasping forceps is entered through the forceps opening 18 to hold the turning unit 40 in the body cavity. Then the introducer 13 is pulled out of the body cavity slowly. The turning unit 40 being withdrawn is abandoned. The supporter 41 is removed from the introducer 13 and then abandoned in a manner similar to the turning unit 40. Note that it is desirable to produce the self-propelling device 11 as a disposable product, and to abandon the same unfailingly after one use irrespective of presence or absence of damage of the turning unit 40.

When the self-propelling device 11 is produced as a reused product, the turning unit 40 being withdrawn is abandoned if broken, as described above. However, the supporter 41 is pre-washed while mounted on the introducer 13. After the pre-washing, the supporter 41 is removed from the introducer 13. Then the supporter 41 and the electronic endoscope 10 are thoroughly washed discretely.

The supporter 41 is disassembled before the thorough washing. After removing the cover 52 from the supporter 41, the pins 61 and 62 are pulled out to remove the follower roller units 55 from the second support sleeve 51 easily. Thus, the second support sleeve 51 can be easily disassembled from the first support sleeve 50. Furthermore, the first support sleeve 50 is removed from the coupling member 66. In short, the supporter 41 is disassembled to wash its elements thoroughly. Note that the drive sleeve 64 is difficult to remove from the coupling member 66 due to the mesh of the pinion 68 with the gear 65. The drive sleeve 64 is put in a container containing washing water together with the coupling member 66, and is washed while the pinion 68 is driven and rotated by the torque wire 67.

After the supporter 41 is assembled by use of the parts having been washed and dried finally, a new turning unit 40 is secured to the supporter 41. Note that, if the self-propelling device 11 is a reused product, the turning unit 40 may be washed while positioned on the supporter 41 on the condition without damage of the turning unit 40. However, it is preferable to remove and abandon the turning unit 40 from the supporter 41 irrespective of presence or absence of the damage, to disassemble and wash the supporter 41 as described above.

In the embodiment described above, the cover of the cylindrical form described above is formed from resin of a rather soft property. However, the present invention is not limited thereto. For example, the cover can be formed from hard resin or metal. For this structure, it is preferable to set an inner diameter of the cover slightly larger than an outer diameter of the second support sleeve, and to fix the cover to the second support sleeve with plural screws after fitting the cover on the outer surface of the second support sleeve. An inlet of screw holes in the cover is provided with a recess for entry of screw heads of the screws, so that the screw heads are prevented from projecting to the outside from the outer surface of the cover. Also, it is possible to use quadrilateral plates as a cover of the invention in a size corresponding to the follower roller units, and to attach the quadrilateral plates to the outer surface of the second support sleeve with screws or the like.

In the above embodiment, the follower roller units are created by unifying a pair of follower rollers. However, the present invention is not limited thereto. For example, three or four follower rollers can be unified. For this structure, two follower rollers adjacent to one another among those follower rollers press the turning unit to the drive wheels in cooperation in a manner similar to the above embodiment. Also, the follower rollers can be discretely pressed on the second support sleeve without using the follower roller units. Also, the follower rollers are disposed in the central portion of the second support sleeve in the longitudinal direction. However, follower rollers can be disposed in plural positions of ends of the second support sleeve for carrying out circulation of the turning unit more smoothly.

In the above embodiment, the curved surfaces 71 and the inclined surfaces 72 are arcuate and curved as viewed in the cross section. However, the inclined surfaces 72 can be conical and straight as viewed in the cross section. Also, the inclined surfaces 72 can be inclined with a gradient different from an end portion of the curved surfaces 71, and may not be continuous with the curved surfaces 71.

In the above embodiment, the turning unit (turning unit of a doughnut form called “toroid”) well-known conventionally is used. However, the present invention is not limited thereto. For example, a turning unit having plural perforations or endless belts can be used as a turning unit. In the use of the turning unit with the perforations, the turning unit can be torn easily with the perforations to facilitate exchange of the turning unit. Although the turning unit with the perforations is more fragile than a normal turning unit, the follower rollers are covered with the cover even upon tearing and prevented from appearing externally. There is no damage of the inner wall of the body cavity.

In the above embodiment, the outer surface 48 and the cover 52 are cylindrical. However, the endless belts may be arranged in a form of a polygonal prism. For this form, the outer surface 48 and the cover 52 can be in a prismatic form.

In use of the belts as the turning unit, the drive wheels are arranged in the three positions in the circumferential direction of the first support sleeve in the example of the above embodiment. So three belts are used totally with one for each of the arrangement positions of the drive wheels. In short, the belts of the number equal to the number of the drive wheels arranged in the circumferential direction of the first support sleeve are used. The belts are formed from material similar to that of the turning unit of the above embodiment. It is preferable to form guides on the first and second support sleeves and the like for preventing the belts from offsetting in the circumferential direction of the first and second support sleeves. It is easily possible to remove the belts from the supporter in the use of the belts as turning unit, because the belts can be cut easily by scissors or the like.

In the above embodiment, the self-propelling device is applied to the electronic endoscope for the medical diagnosis. However, the present invention is not limited to the use in the medical diagnosis, but can be applied to a coupling member for an instrument for imaging of a body cavity, such as an endoscope for industrial use or the like, and an ultrasonic probe.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A self-propelling device comprising:
a cylindrical coupling member for mounting on a tip portion of an endoscope;
a first supporter, formed cylindrically, and fitted on an outer side of said coupling member;
a turning unit, formed from flexible material of a sheet form, for covering an outer surface of said first supporter at least partially;
a cylindrical second supporter, disposed in an inner space defined by an inner surface of said turning unit, for supporting said turning unit from inside;
a follower roller, disposed on said second supporter in a rotatable manner, for contacting an inner surface of said turning unit;
a cover, fitted on an outer surface of said second supporter, for preventing said follower roller from being exposed outside said second supporter;
a drive wheel, disposed on said first supporter, for rotating by nipping said turning unit together with said follower roller, for turning said turning unit to circulate around said second supporter and said cover in a longitudinal direction of said first supporter.

2. A self-propelling device as defined in claim 1, wherein said follower roller is secured so as not to project from said outer surface of said second supporter externally.

3. A self-propelling device as defined in claim 2, wherein said cover is cylindrical to cover said outer surface of said second supporter.

4. A self-propelling device as defined in claim 3, wherein said cover is formed from soft resin.

5. A self-propelling device as defined in claim 1, wherein a plurality of said follower roller are combined in a unit and secured to said second supporter in a removable manner.

6. A self-propelling device as defined in claim 1, wherein said turning unit is in a doughnut form.

7. A self-propelling device as defined in claim 1, wherein said turning unit is an endless belt.
